(19) United States
(12) Patent Application Publication
(10) Pub. No.: US 2013/0261369 A1
(43) Pub. Date: Oct. 3, 2013

(54) TARGET FOR GENERATING ION AND TREATMENT APPARATUS USING THE SAME

(71) Applicant: ELECTRONICS AND TELECOMMUNICATIONS RESEARCH IN, Daejeon (KR)

(72) Inventors: Moon Youn JUNG, Daejeon (KR); Dong-Ho SHIN, Daejeon (KR); Seunghwan KIM, Daejeon (KR)

(73) Assignee: Electronics and Telecommunications Research Institute, Daejeon (KR)

(21) Appl. No.: 13/673,894

(22) Filed: Nov. 9, 2012

(30) Foreign Application Priority Data
Mar. 30, 2012 (KR) 10-2012-0033404

(51) Int. Cl. A61N 5/10; H01J 37/08

(52) U.S. Cl. CPC A61N 5/1077; H01J 37/08
USPC 600/1; 250/423 R

(57) ABSTRACT

Provided are an ion generation target and a treatment apparatus using the same. The treatment apparatus includes an ion generation material generating the ions by incident laser beam, the ion generation material generating a bubble having a hemispheric shape, a support supporting the bubble having the hemispheric shape, a bubble generation member for generating the bubble having the hemispheric shape on the support by using the ion generation material, and a laser radiating laser beam onto a surface of the bubble to generate ions from the ion generation material, thereby projecting the ions onto a tumor portion of a patient.

### CROSS-REFERENCE TO RELATED APPLICATIONS

This U.S. non-provisional patent application claims priority under 35 U.S.C. §119 of Korean Patent Application No. 10-2012-0033404, filed on Mar. 30, 2012, the entire contents of which are hereby incorporated by reference.

### BACKGROUND OF THE INVENTION

The present invention relates to a target for generating ions and a treatment apparatus using the same, and more particularly, to a target for generating protons or carbon ions and an ion beam treatment apparatus using the same.

Methods for radiotherapy may include X-ray treatments, electron beam treatments, and ion beam treatments. Among these, since the X-ray treatments are the cheapest treatment methods which can be realized using the simplest device, the X-ray treatments are being most commonly used at the present day. Although it has been proven in 1950's that tumors can be treated by accelerating electrons using an accelerator to inject the electrons into the tumors, with the miniaturization of an electron accelerator in 1980's, the electron beam treatments have been taken over as one method for radiotherapy. In the X-ray treatments or the electron beam treatments, hydrogen bonds within cancer cells may be cut to destroy the DNA of the cancel cells. However, side effects in which healthy cells existing within the traveling path of the X-ray or electron beam are seriously damaged may be followed. Technologies such as intensity-modulated radiation therapy (IMRT), tomo therapy, cyber knife, and the like have been developed as methods for reducing the radiation exposure of the normal cells. However, the technologies did not completely solve the above-described side effects.

The ion beam treatments are in the spotlight as treatment methods which can mitigate the side effects due to the X-ray treatments or the electron beam treatments. To allow the ion beam to penetrate a material, the ion beam should be accelerated to have high velocity, like the electrons. Even though the ion beam is gradually decreased in velocity when the ion beam penetrates a certain material, the ion beam is subject to the most energy loss of ionizing radiation just before the ion beam is stopped. This phenomenon is called a Bragg peak after William Henry Bragg, which discovered the phenomenon in 1903. Thus, in a case of such an ion beam treatment, malignant tumors may be selectively and locally treated when the ions are precisely controlled in velocity. Also, in a case where the tumors are disposed at a deep position of the human body, protons or ions, each having very high energy, should be accelerated at the outside of the human body. A method for accelerating the protons or ions may include a laser driven ion acceleration method. When high-output laser beam is radiated onto a thin film, ions or protons within the thin film may be escaped with acceleration energy by a target normal sheath acceleration model (TNSA model), a radiation pressure acceleration model (RPA model), or the like. According to a general principle of the ion beam treatment, the escaped ions may penetrate the body of a patient by energy thereof and then be stopped at a predetermined depth at which the tumor is located. Here, a large amount of free oxygen radicals may be generated in the stopped region to necrotize the tumor cells.

A laser for accelerating the ions should have vary high energy of about 10¹⁹ W/cm² to about 10²¹ W/cm². This represents that a very large laser system is required to take a large part of budget.

### SUMMARY OF THE INVENTION

The present invention provides an ion generation target which can generate protons or carbon ions having high energy and an ion beam treatment apparatus using the same.

The feature of the inventive concept is not limited to the aforesaid, but other features not described herein will be clearly understood by those skilled in the art from descriptions below.

Embodiments of the present invention provide ion generation targets including: an ion generation material generating the ions by incident laser beam, the ion generation material generating a bubble having a hemispheric shape; and a support supporting the bubble having the hemispheric shape.

In some embodiments, the ions may be protons or carbon ions.

In other embodiments, the ions may be the protons, and the ion generation material may be water.

In still other embodiments, the ions may be the carbon ions, and the ion generation material may be oil containing a carbon component.

In even other embodiments, the support may be a transparent substrate or a ring type bubble support.

In yet other embodiments, the transparent substrate may be formed of quartz.

In further embodiments, the ring type bubble support may be an opened ring in which a portion thereof is cut off.

In still further embodiments, a thickness of a membrane of the bubble may be adjusted by viscosity of the ion generation material.

In even further embodiments, the ion generation material may further include graphene powder or graphite powder.

In yet further embodiments, the ion generation material may further include an ionizable material.

In other embodiments of the present invention, ion beam treatment apparatuses include: an ion generation target including an ion generation material generating the ions by incident laser beam, the ion generation material generating a bubble having a hemispheric shape and a support supporting the bubble having the hemispheric shape; a bubble generation member for generating the bubble having the hemispheric shape on the support by using the ion generation material; and a laser radiating laser beam onto a surface of the bubble to generate ions from the ion generation material, thereby projecting the ions onto a tumor portion of a patient.

In some embodiments, the support may be a transparent substrate or a ring type bubble support.

In other embodiments, the support may be a transparent substrate, and the bubble generation member may include: an air generation device; and an air delivery tube for providing air generated from the air generation device between the transparent substrate and the ion generation material to generate the bubble. A thickness of a membrane of the bubble may be adjusted by a pressure of the air.

In still other embodiments, the support may be a ring type bubble support, and the bubble generation member may be a wind generation device for blowing wind toward a ring portion of the ring type bubble support. A size of the bubble and a thickness of a membrane of the bubble may be adjusted by a velocity of the wind.

In even other embodiments, the laser and the wind generation device may be respectively disposed on both sides of the ring type bubble support or disposed on one side of the ring type bubble support.

In yet other embodiments, the laser beam may be obliquely incident into a central surface of the bubble.

In further embodiments, the laser beam may be incident into an outer surface or inner surface of the bubble.

In still further embodiments, the laser beam may be a femto-second laser beam.

In even further embodiments, the ion beam treatment apparatuses may further include an optical member for focusing the laser beam onto the surface of the bubble.

In yet further embodiments, the ion beam treatment apparatuses may further include a vacuum chamber including an optical member for focusing the laser beam onto the surface of the bubble. The vacuum chamber may include: an input window through which the laser beam is inputted; the optical member for focusing the laser beam; and an output window through which the focused laser beam is incident into the surface of the bubble. The optical member may include: a plane mirror for reflecting the inputted laser beam; and an off-axis mirror for reflecting and focusing the reflected laser beam at the same time.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is a conceptual view for explaining an ion beam treatment apparatus according to an embodiment of the present invention;

FIGS. 2A and 2B are solid conceptual views for explaining a target for generating ions which is used in the ion beam treatment apparatus according to an embodiment of the present invention;

FIGS. 3 and 4 are solid conceptual views for explaining modified examples of the target for generating ions which is used in the ion beam treatment apparatus according to an embodiment of the present invention; and

FIG. 5 is a conceptual view for explaining an ion beam treatment apparatus according to another embodiment of the present invention.

### DETAILED DESCRIPTION OF PREFERRED EMBODIMENTS

Preferred embodiments of the present invention will be described below in more detail with reference to the accompanying drawings. Advantages and features of the present invention, and implementation methods thereof will be clarified through following embodiments described with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. In the drawings, the dimensions of layers and regions are exaggerated for clarity of illustration.

In the following description, the technical terms are used only for explain a specific exemplary embodiment while not limiting the present invention. The terms of a singular form may include plural forms unless referred to the contrary. The meaning of “include,” “comprise,” “including,” or “comprising,” specifies a property, a region, a fixed number, a step, a process, an element and/or a component but does not exclude other properties, regions, fixed numbers, steps, processes, elements and/or components. Since preferred embodiments are provided below, the order of the reference numerals given in the description is not limited thereto. It will be understood that when an element such as a layer, film, region, or substrate is referred to as being “on” another element, it can be directly on the other element or intervening elements may also be present.

Additionally, the embodiment in the detailed description will be described with sectional views as ideal exemplary views of the present invention. In the figures, the dimensions of layers and regions are exaggerated for clarity of illustration. Accordingly, shapes of the exemplary views may be modified according to manufacturing techniques and/or allowable errors. Therefore, the embodiments of the present invention are not limited to the specific shape illustrated in the exemplary views, but may include other shapes that may be created according to manufacturing processes. For example, an etched region illustrated or described as a rectangle will, typically, have rounded or curved features. Thus, the regions illustrated in the figures are schematic in nature and their shapes are not intended to illustrate the precise shape of a region of a device and are not intended to limit the scope of the present invention.

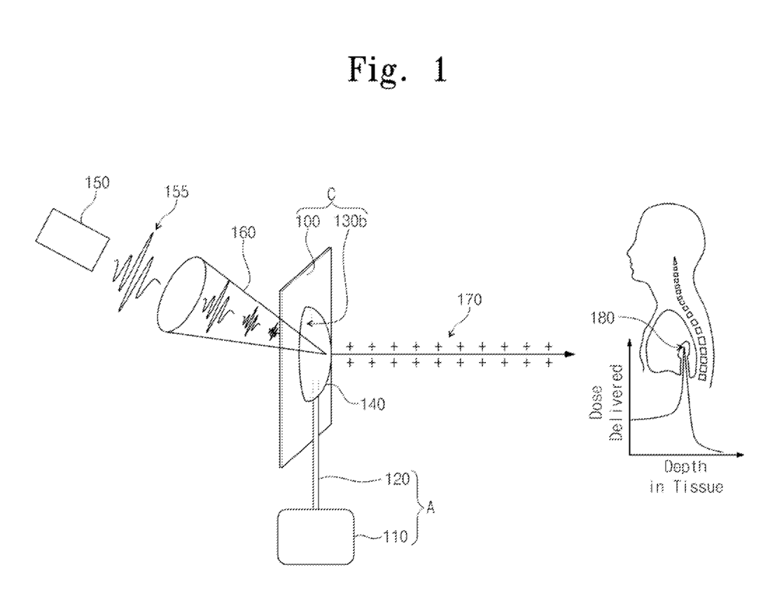

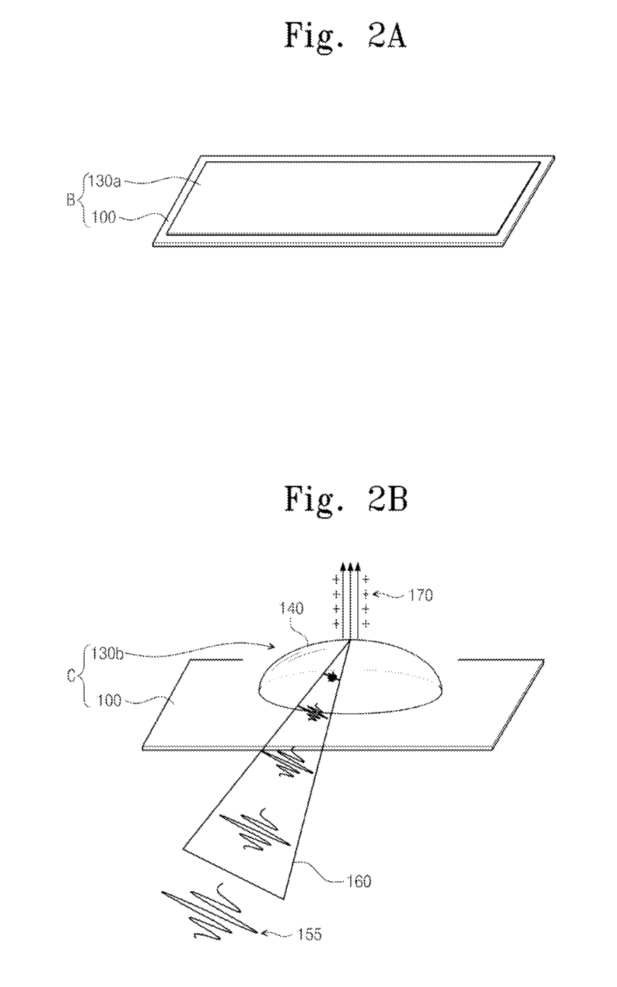

FIG. 1 is a conceptual view for explaining an ion beam treatment apparatus according to an embodiment of the present invention. FIGS. 2A and 2B are solid conceptual views for explaining a target for generating ions which is used in the ion beam treatment apparatus according to an embodiment of the present invention.

Referring to FIGS. 1, 2A, and 2B, an ion beam treatment apparatus includes a laser 150, an optical member 160, a target C for generating ions (hereinafter, referred to as an “ion generation target C”), and a bubble generation member A.

The laser 150 may generate ions 170 from the ion generation target C to project the ions into a tumor portion 180 of a patient. The laser 150 may provide laser beam 155 into the ion generation target C. The laser beam 150 may be femto-second laser beam.

The ion generation target C may receive the laser beam 155 from the laser 150 to generate the ions 170. The ion generation target C may include an ion generation material 130a for generating a bubble having a hemispheric shape and a transparent substrate 100 for supporting the bubble 130b with the hemispheric shape. The transparent substrate 100 may include a quartz substrate having high transmittance with respect to the laser beam 155. The ion generation material 130a may generate ions by the incident laser beam 155. The ions 170 may be protons or carbon ions.

A membrane of the bubble 130b may have a thickness of about several hundred nanometers (nm) to about several microns (μm). The thickness of the membrane of the bubble 130b may be adjusted by viscosity of the ion generation material 130a.

When the ions 170 are protons, the ion generation material 130a may be pure water. Also, when the ions 170 are carbon ions, the ion generation material 130a may be oil containing a carbon component.

Graphene powder or graphite powder which are supply sources of the carbon ions may be further provided on a surface 140 of the bubble 130b. Also, an ionizable material may be further provided on the surface 140 of the bubble 130b. The ionizable material may include Na, S, and Mg.

The optical member 160 may focus the laser beam 155 onto the surface 140 of the bubble having the hemispheric shape. The optical member 160 may increase intensity of the laser beam 155 so that the laser beam has intensity of about 10¹⁹ W/cm² or more. The optical member 160 for focusing the laser beam 155 may be an off-axis parabola mirror. Thus, the ions 170 generated from the bubble 130b of the ion generation target C may be protons or carbon ions having high energy of about several tens MeV to several hundreds MeV. That is, the ions 170 generated from the bubble 130b of the ion generation target C may have energy adjusted by the intensity of the laser beam 155. Thus, the ions 170 may be stopped at the tumor portion 180 of the patient to collide with the tumor portion 180.

The bubble generation member A may include an air generation device 110 and an air delivery tube 120 disposed between the transparent substrate 100 and the ion generation material 130a. A size of the bubble 130b and a thickness of the membrane of the bubble 130b of the ion generation target C may be adjusted by a pressure of air generated by the bubble generation member A.

When the femto-second laser beam is precisely focused onto the surface 140 of the bubble 130b of the ion generation target C, the ions 170 may be accelerated by a target normal sheath acceleration model (TNSA model) or a radiation pressure acceleration model (RPA model). The laser beam 155 may be obliquely incident into a central surface of the bubble 130b of the ion generation target C. This is done because the laser 150 may be damaged by a portion of the laser beam 155 reflected from the surface 140 of the bubble 130b when the laser beam 155 is focused into a front central surface of the bubble 130b of the ion generation target C.

As shown in FIGS. 2A and 2B, when air is provided from the bubble generation member A into a preliminary ion generation target B including the ion generation material 130a on the transparent substrate 100, the ion generation target C having a plurality of bubbles may be provided on the transparent substrate 100. Here, the laser beam 155 may be focused onto the surface 140 of the outermost bubble 130b of the ion generation target C.

The laser beam 155 may be incident from an inner surface of the bubble 130b of the ion generation target C into an outer surface. The ions 170 may be accelerated and ejected in a direction in which the laser beam 155 is incident into the ion generation target C.

The ions 170 may be set to be projected onto a position of the tumor portion 180 obtained from image diagnosis equipment such as a magnetic resonance imaging (MRI), a computer tomography (CT), a positron emission tomography (PET), or ultrasonics wave equipment which are apparatuses used for diagnosing the tumor portion 180 of the patient.

According to a treatment principle of the ion beam treatment apparatus, the laser beam 155 provided from the laser 150 is provided onto the surface 140 of the bubble 130b of the ion generation target C to generate the ions 170 from the bubble 130b. Then, the ions 170 are projected onto the inside of the body of the patient. The ions 170 projected onto the inside of the body of the patient are stopped at the tumor portion 180 within the body of the patient by a Bragg peak principle to collide with the tumor portion 180. As a result, the ions 170 generate free oxygen radicals to disturb tumor cells of the tumor portion 180.

That is, since the ions 170 collide with the tumor portion 180 to generate the free oxygen radicals and disturb the tumor cells of the tumor portion 180, growth of the tumor cells may be thwarted or the tumor cells may be necrotized. Here, the disturbance of the tumor cells of the tumor portion 180 by the ions 170 may represents disturbance of DNA double helices of the tumor cells or disturbance of a metabolic process within nuclei of the tumor cells.

The generation and projection processes of the ions 170 in the ion generation target C of the ion beam treatment apparatus will be described in more detail with reference to FIGS. 1, 2A, and 2B.

According toe the generation and projection processes of the ions 170, when the laser beam 155 is incident into the surface 140 of the bubble 130b of the ion generation target C, hydrogen atoms or carbon ions contained in the bubble 130b are changed in a plasma state in which the hydrogen atoms or carbon ions are divided into positive ions 170 and negative ions (not shown) by energy of the laser beam 155. In this process, the negative ions further come away from the bubble 130b than the positive ions 170 to generate electric fields by a capacitor effect between the positive ions 220 and the negative ions. Thus, the positive ions 170 are accelerated toward the negative ions by the electric fields so that each of the positive ions 170 has energy enough to be projected onto the tumor portion 180 of the inside of the body of the patient from the outside of the body.

The accelerated ions 170 collide with the tumor portion 180 within the body of the patient to generate the free oxygen radicals and disturb the tumor cells of the tumor portion 180. Thus, the tumor cells may be thwarted in growth or necrotized. Therefore, the tumor portion 180 within the body of the patient may be treated.

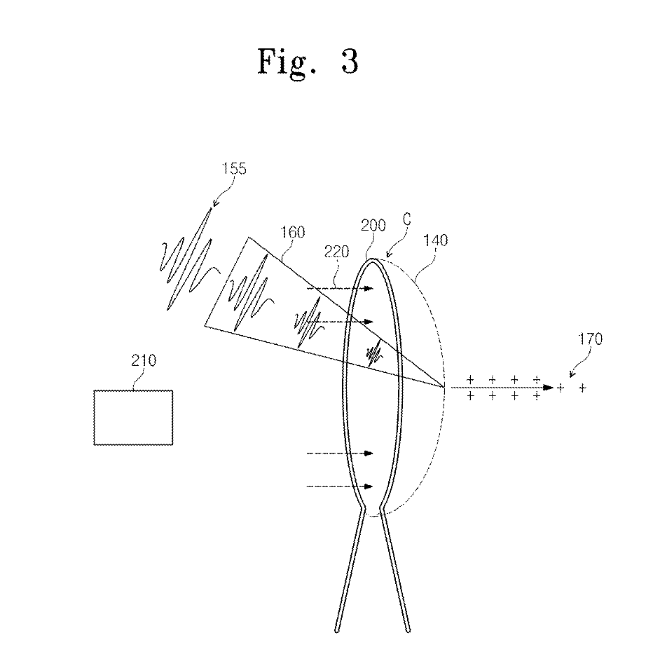

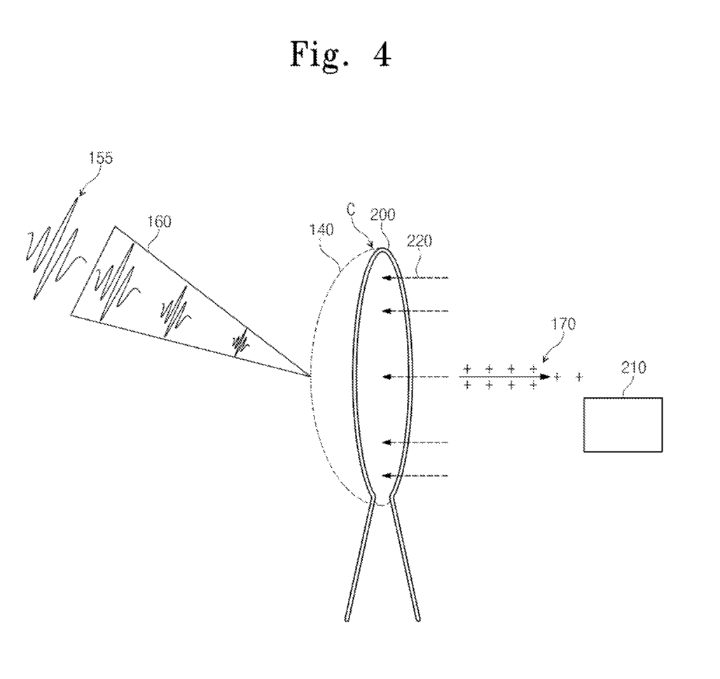

FIGS. 3 and 4 are solid conceptual views for explaining modified examples of the target for generating ions which is used in the ion beam treatment apparatus according to an embodiment of the present invention. For convenience of description, the explanation of the same or similar portions to the description in FIGS. 1, 2A, and 2B will be omitted.

Referring to FIGS. 3 and 4, an ion beam treatment apparatus includes a laser (see reference number 1 of FIG. 1), an optical member 160, an ion generation target C, and a wind generator 210.

The ion generation target C may receive laser beam 155 from a laser to generate ions 170. The ion generation target C may include an ion generation material (see reference number 130a of FIG. 2A) for generating a bubble (see reference number 130b of FIG. 1) having a hemispheric shape and a ring type bubble support 220 for supporting the bubble having the hemispheric shape. As shown in FIGS. 3 and 4, the ring type bubble support 200 may be an opened ring in which a portion thereof is cut off, but a closed ring. This is done for easily installing the ring type bubble support 200. Thus, the present invention is not limited thereto. For example, a ring type bubble support having the closed ring structure may be used as the ring type bubble support 200. The ion generation material may generate ions by the incident laser beam 155. The ions 170 may be protons or carbon ions.

A membrane of the bubble may have a thickness of about several hundred nanometers (nm) to about several microns (μm). A thickness of the membrane of the bubble may be adjusted by viscosity of the ion generation material.

When the ions 170 are protons, the ion generation material may be pure water. Also, when the ions 170 are carbon ions, the ion generation material may be oil containing a carbon component.

Graphene powder or graphite powder which are supply sources of the carbon ions may be further provided on a surface 140 of the bubble. Also, an ionizable material may be further provided on the surface 140 of the bubble. The ionizable material may include Na, S, and Mg.

The optical member 160 may focus the laser beam 155 onto the surface 140 of the bubble having a hemispheric shape. The optical member 160 may increase intensity of the laser beam 155 so that the laser beam has intensity of about 10¹⁹ W/cm² or more. The optical member 160 for focusing the laser beam 155 may be an off-axis parabola mirror. Thus, the ions 170 generated from the bubble of the ion generation target C may be protons or carbon ions having high energy of about several tens MeV to several hundreds MeV.

The wind generator 210 may blow wind 220 toward a ring portion of the ring type bubble support 200. A size of the bubble and a thickness of the membrane of the bubble may be adjusted by velocity of the wind 220 blown from the wind generator 210.

As shown in FIG. 3, all of the laser and the wind generator 210 may be disposed on one side of the ring type bubble support 200. Here, the laser beam 155 may be incident into an inner surface of the bubble of the ion generation target C. On the other hand, as shown in FIG. 4, the laser and the wind generator 210 may be disposed on both sides of the ring type bubble support 200, respectively. Here, the laser beam 155 may be incident into an outer surface of the bubble of the ion generation target C. The ions 170 may be accelerated and ejected in a direction in which the laser beam 155 is incident into the ion generation target C.

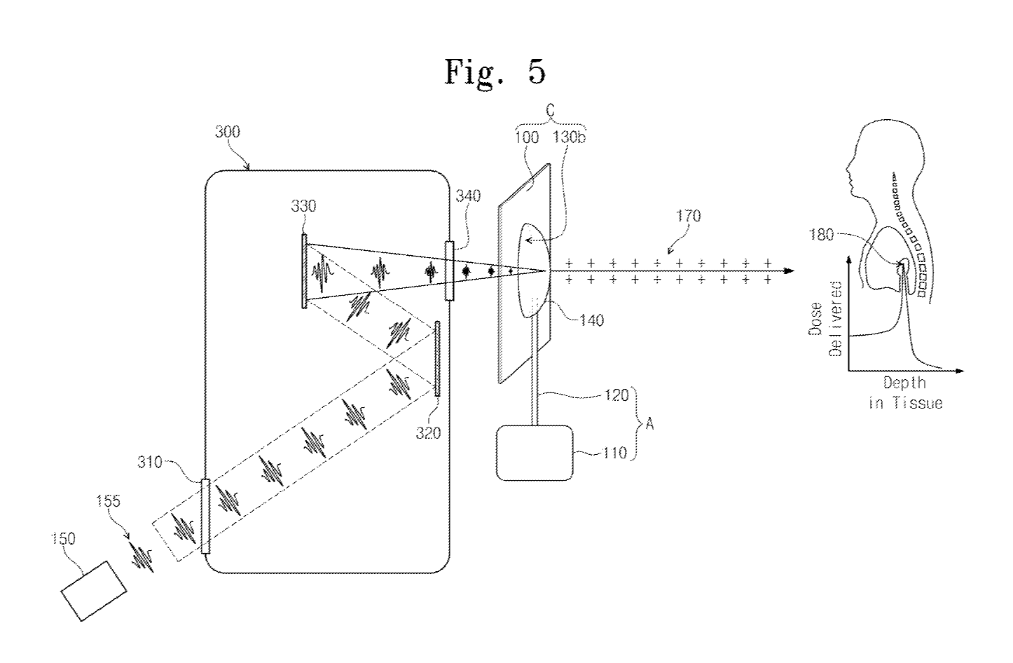

FIG. 5 is a conceptual view for explaining an ion beam treatment apparatus according to another embodiment of the present invention. For convenience of description, the explanation of the same or similar portions to the description in FIGS. 1, 2A, and 2B will be omitted.

Referring to FIG. 5, an ion beam treatment apparatus includes a laser 150, a vacuum chamber 300 including optical members 320 and 330 therein, an ion generation target C, and a bubble generation member A.

The vacuum chamber 300 may include an input window 310 through which laser beam 155 is inputted, the optical members 320 and 330 for focusing the laser beam 155, and an output wind 340 through which the laser beam 155 focused onto a surface 140 of a bubble 130b of the ion generation target C is incident.

The optical members 320 and 330 may include a plane mirror 320 for reflecting the inputted laser beam 155 and an off-axis mirror 330 for reflecting and focusing the reflected laser beam 155 at the same time. Preferably, the off-axis mirror 330 may be an off-axis parabola mirror.

That is, the laser beam 155 generated from the laser 150 is introduced into the vacuum chamber 300 through the input window 310, reflected by the plane mirror 320, reflected by the off-axis mirror 330, and focused onto the surface 140 of the bubble 130b of the ion generation target C through the output window 340. Thus, the ions 170 generated from the bubble 130b of the ion generation target C may be protons or carbon ions having high energy of about several tens MeV to several hundreds MeV.

The vacuum chamber 300 may minimize reduction of the intensity of the laser beam when the laser beam 155 proceeds into air.

Since the ion generation target according to the embodiments of the present invention includes an ultra thin film having the bubble shape, the protons and carbon ions having the high energy may be generated. Thus, a high process technology such as a semiconductor processing process for manufacturing the ultra thin film may be not required. Thus, the ion generation target including the ultra thin film may be provided without taking the accompanying high costs.

Also, since the ion beam treatment apparatus according to the embodiments of the present invention uses the ion generation target including the ultra thin film having the bubble shape, the protons and carbon ions having the high energy may be projected onto the tumor portion of the patient. Therefore, the ion beam treatment apparatus which can treat the tumor portion of the patient at low costs may be provided.

As described above, since the ion generation target according to the embodiments of the present invention includes the ultra thin film having the bubble shape, the protons and carbon ions having the high energy can be generated. Thus, a high process technology such as a semiconductor processing process for manufacturing the ultra thin film may be not required. Thus, the ion generation target having the ultra thin film shape can be provided without taking the accompanying high costs.

Also, since the ion beam treatment apparatus according to the embodiments of the present invention uses the ion generation target including the ultra thin film having the bubble shape, the protons and carbon ions having the high energy can be projected onto the tumor portion of the patient. Therefore, the ion beam treatment apparatus which can treat the tumor portion of the patient at low costs can be provided.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. An ion generation target comprising:
an ion generation material generating the ions by incident laser beam, the ion generation material generating a bubble having a hemispheric shape; and
a support supporting the bubble having the hemispheric shape.

2. The ion generation target of claim 1, wherein the ions are protons or carbon ions.

3. The ion generation target of claim 2, wherein the ions are the protons, and the ion generation material is water.

4. The ion generation target of claim 2, wherein the ions are the carbon ions, and the ion generation material is oil containing a carbon component.

5. The ion generation target of claim 1, wherein the support is a transparent substrate or a ring type bubble support.

6. The ion generation target of claim 1, wherein a thickness of a membrane of the bubble is adjusted by viscosity of the ion generation material.

7. The ion generation target of claim 1, wherein the ion generation material further comprises graphene powder or graphite powder.

8. An ion beam treatment apparatus comprising:
an ion generation target comprising an ion generation material generating the ions by incident laser beam, the ion generation material generating a bubble having a hemispheric shape and a support supporting the bubble having the hemispheric shape;
a bubble generation member for generating the bubble having the hemispheric shape on the support by using the ion generation material; and
a laser radiating laser beam onto a surface of the bubble to generate ions from the ion generation material, thereby projecting the ions onto a tumor portion of a patient.

9. The ion beam treatment apparatus of claim 8, wherein the support is a transparent substrate or a ring type bubble support.

10. The ion beam treatment apparatus of claim 9, wherein the support is a transparent substrate, and
the bubble generation member comprises:
an air generation device; and
an air delivery tube for providing air generated from the air generation device between the transparent substrate and the ion generation material to generate the bubble.

11. The ion beam treatment apparatus of claim 10, wherein a thickness of a membrane of the bubble is adjusted by a pressure of the air.

12. The ion beam treatment apparatus of claim 9, wherein the support is a ring type bubble support, and
the bubble generation member is a wind generation device for blowing wind toward a ring portion of the ring type bubble support.

13. The ion beam treatment apparatus of claim 12, wherein a size of the bubble and a thickness of a membrane of the bubble are adjusted by a velocity of the wind.

14. The ion beam treatment apparatus of claim 8, wherein the laser beam is obliquely incident into a central surface of the bubble.

15. The ion beam treatment apparatus of claim 8, wherein the laser beam is incident into an outer surface or inner surface of the bubble.

16. The ion beam treatment apparatus of claim 8, wherein the laser beam is a femto-second laser beam.

17. The ion beam treatment apparatus of claim 8, further comprising an optical member for focusing the laser beam onto the surface of the bubble.

18. The ion beam treatment apparatus of claim 8, further comprising a vacuum chamber comprising an optical member for focusing the laser beam onto the surface of the bubble.

19. The ion beam treatment apparatus of claim 18, wherein the vacuum chamber comprises:
an input window through which the laser beam is inputted;
the optical member for focusing the laser beam; and
an output window through which the focused laser beam is incident into the surface of the bubble.

20. The ion beam treatment apparatus of claim 19, wherein the optical member comprises:
a plane mirror for reflecting the inputted laser beam; and
an off-axis mirror for reflecting and focusing the reflected laser beam at the same time.
